(19) United States
(12) Patent Application Publication
(10) Pub. No.: US 2025/0049466 A1
(43) Pub. Date: Feb. 13, 2025

(54) SAFETY THROMBECTOMY DEVICE

(71) Applicant: BEIJING HONGHAI MICROTECH CO., LTD., Beijing (CN)

(72) Inventors: Xuemin ZHANG, Beijing (CN); Xunming JI, Beijing (CN); Tao WANG, Beijing (CN); Jianliang ZHENG, Beijing (CN); Dongdong CHEN, Beijing (CN); Xuzhi ZHANG, Beijing (CN); Yan WU, Beijing (CN); Ming LI, Beijing (CN)

(21) Appl. No.: 18/711,222

(22) PCT Filed: Nov. 21, 2022
(86) PCT No.: PCT/CN2022/133202

§ 371 Date: May 17, 2024

(30) Foreign Application Priority Data
Nov. 25, 2021 (CN) 202111417736.5

(51) Int. Cl. A61B 17/3207; A61B 17/22; A61B 17/221

(52) U.S. Cl. CPC A61B 17/320725; A61B 17/221; A61B 2017/22064

(57) ABSTRACT

A safety thrombectomy device, comprising: a handle (1); a hollow outer tube (2); a hollow inner core tube (3) having a first inner core segment located in a through hole in the outer tube (2), a second inner core segment extending from the front end of the outer tube (2), and a third inner core segment extending from the rear end of the outer tube (2); a thrombus crusher (5) provided at the front end of the outer tube (2); a thrombus collector (4) connected to the front end of the second inner core segment of the hollow inner core tube (3) and located in front of the thrombus crusher (5); a tee joint (6) provided at the front end of the handle (1) and having a free end (10) used for injecting normal saline; an adjusting knob (7) provided on the handle (1) and capable of moving with respect to the handle (1) to drive the outer tube (2) to move back and forth; a locking knob (8) provided on the handle (1) and used for locking the adjusting knob (7) after the outer tube (2) moves to a preset position; and an injector (9) provided on the third inner core segment of the hollow inner core tube (3) provided at the rear end of the handle (1) and used for injecting normal saline.

### TECHNICAL FIELD

The present invention relates to a medical device, in particular to a safety thrombectomy device for taking out the thrombus in the blood vessels of the human body from the human body by breaking and collecting it.

### BACKGROUND ART

Thrombosis is a common disease of cardio-cerebral vessels in the human body at present, which is very harmful to human health. The main treatment methods are drug-induced thrombolysis and thrombus removal. At present, the commonly used method to remove thrombus is through interventional therapy, that is, a thrombus crusher and thrombus collector located on a retractable multilayer hose are placed into the position of human thrombosis through blood vessels. The thrombus is crushed into smaller clots by a thrombus crusher, and then collected by a thrombus collector, and then taken out of the human body; The above thrombus crushing and thrombus collection thrombectomy device has two defects in its long-term use. One is that there is air in the multi-layer hose of the thrombectomy device. When it is placed into the human body, the air will enter the blood vessels of the human body to form bubbles, and the air bubble is essentially similar to the thrombus. With the flow of blood, it randomly enters some vascular positions and blocks the flow of blood. The second is that there are smaller thrombus blocks in the process of thrombus crushing by the thrombus crusher. if the collection efficiency of the thrombus collector is not high, the smaller thrombus blocks will escape from the collecting mesh of the thrombus collector and flow into smaller blood vessels with the flow of blood to form new blockages that are more difficult to deal with or treat, especially into the blood vessels of the brain, resulting in more serious consequences. The above-mentioned two defects have existed for many years, the above-mentioned two new blockages still cannot be avoided by the doctor's careful operation. Therefore, how to solve the above-mentioned technical defects in the prior art and improve the safety of thrombectomy device is still of great significance.

### SUMMARY OF THE INVENTION

It is an object of the present invention to provide a thrombectomy device with high safety.

A safety thrombectomy device of the present invention comprises: a handle; a hollow outer tube; a hollow inner core tube having a first inner core segment located in a through hole in the outer tube, a second inner core segment extending from the front end of the outer tube, and a third inner core segment extending from the rear end of the outer tube; a thrombus crusher provided at the front end of the outer tube; a thrombus collector connected to the front end of the second inner core segment of the hollow inner core tube and located in front of the thrombus crusher; a tee joint provided at the front end of the handle and having a free end used for injecting normal saline; an adjusting knob provided on the handle and capable of moving with respect to the handle to drive the outer tube to move back and forth; a locking knob provided on the handle and used for locking the adjusting knob after the outer tube moves to a preset position; and an injector provided on the third inner core segment of the hollow inner core tube provided at the rear end of the handle and used for injecting normal saline.

Preferably, the second inner core segment of the hollow inner core tube extending from the front end of the outer tube passes through the thrombus crusher and the thrombus collector in turn and is connected with the front end of the thrombus collector, so that the thrombus crusher and the thrombus collector can be expanded or tightened when the adjusting knob drives the outer tube to move back and forth.

Preferably, the front end of the second inner core segment of the hollow inner core tube is connected to the first hollow metal ring and the front end of the thrombus collector via the first hollow metal ring.

Preferably, the outer tube is connected to the tee joint and the adjusting knob is connected via the tee joint.

Preferably, the adjusting knob includes a first portion disposed within the handle chute and a second portion extending from the front end of the handle and connected to the tee joint.

Preferably, the front end of the thrombus crusher is connected to the rear end of the thrombus collector by a restraining ring.

Preferably, the first hollow metal ring is connected to a hollow flexible lead-in head in front of it.

Preferably, the safety thrombectomy device of the present invention further comprises a guide wire passing sequentially through the hollow cavity of the hollow inner core tube, the hollow cavity of the first hollow metal ring, and the hollow cavity of the hollow flexible lead-in head.

Preferably, the free end of the tee joint is provided with a plugging end; the outer end of the injector is provided with a plugging end.

Preferably, the front end of the thrombus collector is fixedly connected to the front end of the hollow inner core tube, the rear end of the thrombus crusher is connected to the front end of the outer tube, and the rear end of the thrombus collector is connected to the front end of the thrombus crusher via a restraining ring.

Preferably, the thrombus collector has at least one convex ring at an axially central position of the collecting mesh, the diameter of the convex ring being 0.5-2 mm larger than the diameter of the rest of the collecting mesh.

Preferably, the braiding density of the metal wires of the convex rings is greater than the braiding density of the rest of the collecting mesh.

Preferably, the thrombus collector has two convex rings on the collecting mesh.

Preferably, the thrombus crusher has a crushing mesh made of metal braid wires, the structure of the crushing mesh is that the middle is a hollow cylindrical part, both ends of the hollow cylindrical part are conical parts, wherein the conical part with higher braiding density has a flat section of 1-2 mm, the flat section is close to the edge of the cylindrical part.

The thrombectomy device defined in the above-mentioned technical solution of the present invention, when in use, injects normal saline through the injector at the end of the tee joint and the end of the hollow inner core tube, exhausts the air between the hollow inner core tube and the outer tube and in the hollow through hole of the hollow inner core tube and then inserts same into the human body to crush, collect and take out thrombi, and at the same time, the thrombus collector with a specific design can avoid the escape of thrombi fragments, which greatly improves the safety of the thrombectomy device of the present invention and avoids the safety risks existing in the prior art.

### BRIEF DESCRIPTION OF THE DRAWINGS

FIG. 1 is an overall structural schematic view of an embodiment of the present invention;

FIG. 2 is a view of the thrombectomy device of FIG. 1 in use:

FIG. 3 is an enlarged schematic view of the handle:

FIG. 4 is an enlarged schematic view of a thrombus crusher and thrombus collector:

FIG. 5 is an enlarged schematic view of the connection of the hollow inner core tube to the thrombus crusher and thrombus collector.

### DETAILED DESCRIPTION OF THE INVENTION

A detailed description of specific embodiments of the present invention will be given with reference to the accompanying drawings. It should be noted that the detailed description of the present invention is provided to facilitate an understanding of the technical spirit of the present invention, and should not be construed to limit the scope of the claims of the present invention.

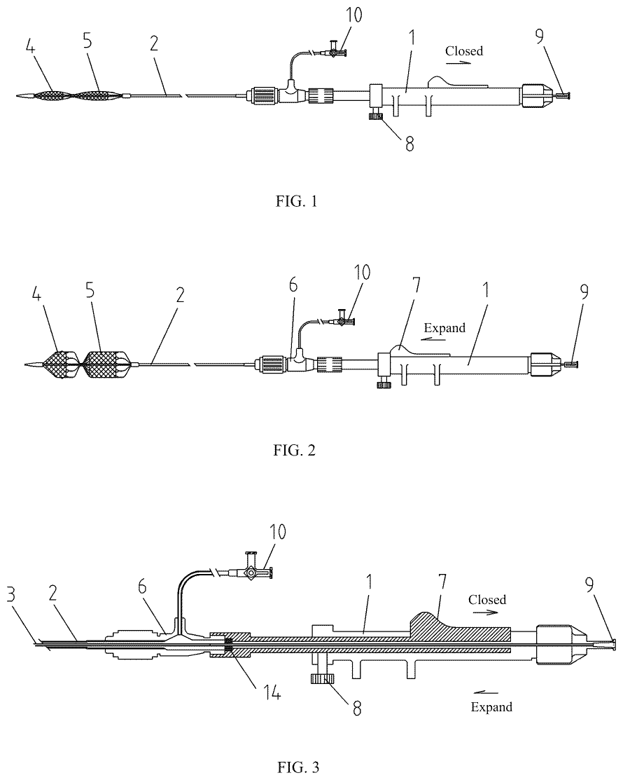

Referring to FIGS. 1-5, a safety thrombectomy device of the present invention includes: a hollow outer tube 2; a hollow inner core tube 3 having a first inner core segment located in a through hole in the outer tube 2, a second inner core segment extending from the front end of the outer tube 2, and a third inner core segment extending from the rear end of the outer tube 2; a thrombus crusher 5 provided at the front end of the outer tube 2; a thrombus collector (4) connected to the front end of the second inner core segment and located in front of the thrombus crusher 5; a tee joint 6 provided at the front end of the handle 1 and having a free end 10 used for injecting normal saline; an adjusting knob 7 provided on the handle 1 and capable of moving with respect to the handle 1 to drive the outer tube 2 to move back and forth; a locking knob 8 provided on the handle 1 and used for locking the adjusting knob 7 after the outer tube 2 moves to a preset position; and an injector 9 provided on the third inner core segment of provided at the rear end of the handle 1 and used for injecting normal saline.

As shown in FIG. 5, the second inner core segment of the hollow inner core tube 3 extending from the front end of the outer tube 2 passes through the thrombus crusher 5 and the thrombus collector 4 in turn and is connected the front end of the thrombus collector 4, so that the thrombus crusher 5 and the thrombus collector 4 can be expanded (as shown in FIG. 2) or tightened (as shown in FIG. 1) when the adjusting knob 7 drives the outer tube 2 to move back and forth. The front end of the second inner core segment of the hollow inner core tube 3 is connected to the first hollow metal ring 16 and the front end of the thrombus collector 4 via the first hollow metal ring 16.

As shown in FIGS. 1 to 3, the outer tube 2 is connected to a tee joint 6 and an adjusting knob 7 via the tee joint 6. As shown in FIG. 3, the adjusting knob 7 includes a first portion disposed within the handle 1 chute and a second portion extending from the front end of the handle 1 and connected to the tee joint 6.

As shown in FIG. 5, the front end of the thrombus crusher 5 is connected to the rear end of the thrombus collector 4 by a restraining ring 18, so that the thrombus crusher 5 and the thrombus collector 4 can be moved together when the outer tube 2 moves back and forth.

As shown in FIG. 5, the present invention also has a hollow flexible lead-in head (15) connected to the first hollow metal ring (16) so that a guide wire (not shown in the figure) for breaking thrombus passes through the hollow cavity of the hollow inner core tube (3), the hollow cavity of the first hollow metal ring (16) and the hollow flexible lead-in head (15) and then extend out when necessary.

In particular, there is a gap between the hollow inner core tube 3 and the outer tube 2 so that the outer tube 2 can move relative to the hollow inner core tube 3. The front end of the handle 1 is provided with a tee joint 6, the through tube of the tee joint 6 is provided with an outer tube 2 and a hollow inner core tube 3, and the rear end of the third inner core segment of the hollow inner core tube 3 are connected with the injector 9 and are fixedly connected to the rear end of the handle 1. An adjusting knob 7 on the handle 1 controls the back-and-forth movement displacement of the adjustment outer tube 2 and the hollow inner core tube 3, and a locking knob 8 can lock the adjusting knob 7 on the handle 1. Before the above-mentioned thrombectomy device is placed into a human blood vessel, normal saline is injected through a free end 10 of the tee joint 6 and the injector 9, the normal saline discharges air in the gap between the outer tube 2 and the hollow inner core tube 3 and air of the hollow channel of the hollow inner core tube 3. A scaling ring 14 is provided at the connection of the tee joint 6 and the adjusting knob 7 to prevent the normal saline from leaking out. After the thrombectomy device is placed into the human blood vessel, the thrombus collector 4 and the thrombus crusher 5 are unfolded along with the displacement of the outer tube 2 and delivered to the thrombectomy position in the blood vessel to perform the operations of crushing and collecting thrombectomy. During the whole operation process, since the air between the outer tube 2 and the hollow inner core tube 3 and air of the hollow cavity of the hollow inner core tube 3 has been previously discharged, the possibility of air entering into a human blood vessel is eliminated, the possibility of forming a new intravascular blockage due to air bubbles entering a human blood vessel is completely avoided, and the safety of thrombectomy is greatly improved.

In order to further improve the safety; the free end 10 of the tee joint 6 is provided with a plugging end, or/and the outer end of the injector 9 is provided with a plugging end (the plugging end is not shown in the figure), wherein the above-mentioned plugging end can close the opening of the free end 10 and the injection port of the injector 9 after the thrombectomy device is injected with normal saline, so as to avoid the escape of normal saline, and air which may exist when the thrombectomy device is repeatedly operated enters the gap between the outer tube 2 and the hollow inner core tube 3 and the hollow cavity of the hollow inner core tube 3 again, ensuring the safety of the thrombectomy process.

The front end of the thrombus collector 4 is provided with a flexible introduction head 15 and a first metal ring 16, the flexible introduction head 15 is connected with the first metal ring 16, and the first metal ring 16 is fixedly connected with the front end of the hollow inner core tube 3; The rear end of the thrombus crusher 5 is connected with the front end of the outer tube 2 and the second metal ring 17, the rear end of the thrombus collector 4 is connected with the front end of the thrombus crusher 5 via a restraining ring 18, and the restraining ring 18 can be displaced along the hollow inner core tube 3. By displacing the outer tube 2 by means of the adjusting knob 7, the thrombus crusher 5 and the thrombus collector 4 are released (expand) or contracted. The thickness of the human blood vessel is monitored, and when the thrombus crusher 5 and the thrombus collector 4 are adapted to the diameter of the blood vessel, the locking knob 8 is rotated, so that the outer tube 2 can no longer be axially displaced, and the operation of breaking the thrombus can be performed. The thrombus collector 4 and the thrombus crusher 5 move together, which can better control release degree and facilitate of operation.

In order to improve the developing ability, at least three or more developing rings 13 are respectively arranged on the thrombus collector 4 and the thrombus crusher 5, and the developing rings 13 are made of a material capable of developing under X-ray irradiation so that the actual expanded and contracted states of the thrombus collector 4 and the thrombus crusher 5 can be clearly observed during the insertion of the thrombus collector 4 and the thrombus crusher 5 into a human blood vessel.

In another example, the hollow inner core tube 3 may sequentially pass through the first metal ring 16 and the hollow cavity of the flexible introduction head 15, and at least the first metal ring 16 may be fixedly connected so as to destroy the thrombus by using a guide wire (not shown) passing through the hollow cavity of the hollow inner core tube 3 so that the contracted or tightened thrombus collector 4 and the thrombus crusher 5 pass through the blood vessel having the thrombus when the contracted or tightened thrombus collector 4 and the thrombus crusher 5 cannot pass through the thrombus as shown in FIG. 1.

During practical use, the thrombus crusher 5 undergoes axial displacement and rotation, the metal braid mesh of the thrombus crusher 5 performs axial and radial cutting on the thrombus, and the thrombus block formed by the cutting is collected by the collecting mesh of the thrombus collector 4. When breaking the thrombus, it is necessary to repeatedly make the thrombus collector 4 and the thrombus crusher 5 move axially and/or rotationally, so that the collecting mesh of the thrombus collector 4 moves to cause deformation of the collecting mesh. The deformation may cause a gap between the collecting mesh and the wall surface of the blood vessel, and the broken thrombus blocks enters the blood vessel through the above-mentioned gap. With the blood circulation, the broken thrombus blocks produce a new smaller blockage of the vessel. In order to avoid the occurrence of the above-mentioned gap, at least one convex ring 11 is provided at position of the collecting mesh of the thrombus collector 4, and the diameter of the convex ring 11 is 0.5-2 mm greater than the diameter of the remaining part of the collecting mesh; Since the collecting net is formed by metal braid wires, the collecting net has a certain elasticity in the radial direction, when the collecting mesh is in a human blood vessel, the elastic deformation and the elastic force received by the convex ring 11 are greater than the rest of the collecting net, and when the collecting net is axially displaced or rotated to generate an integral deformation, the convex ring 11 has a larger radial deformation amount, so that the convex ring 11 maintains a proper contact with the wall surface of the blood vessel, and the above-mentioned gap is avoided, and thus the broken thrombus block is avoided to re-enter the blood circulation, and the safety of the thrombectomy device is further improved.

In order to further improve the safety, the braiding density of the metal wires of the above-mentioned convex rings 11 of the collecting net is greater than the braiding density of the rest of the collecting net, the radial elasticity of the convex rings 11 is further appropriately improved, so that the above-mentioned gap is avoided, and there are two convex rings 11 on the collecting mesh, so that the plugging effect of the convex rings 11 is further improved.

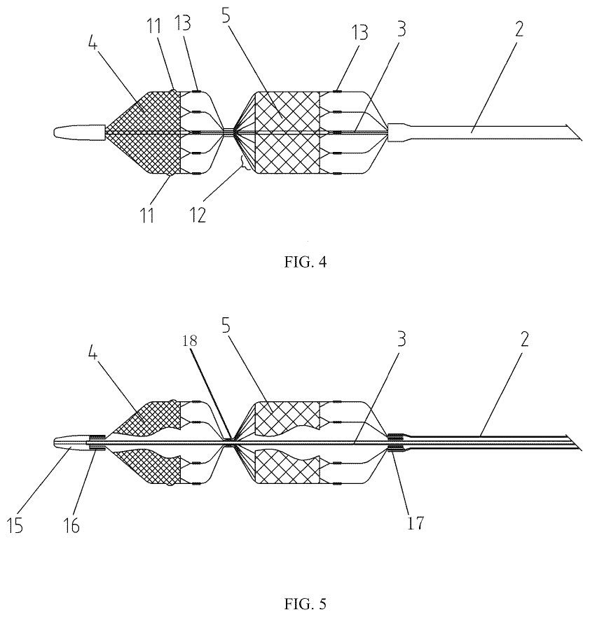

The thrombus crusher 5 is a crushing mesh composed of a metal braid wire, wherein the crushing mesh comprises a cylindrical middle part, a front-end conical part located at the front end of the cylindrical middle part and a rear-end conical part located at the rear end of the cylindrical middle part (see FIGS. 4 and 5). The braid wire of the front-end conical part serves to axially cut the thrombus, the braid wire of the cylindrical middle part mainly performs radial cutting on the thrombus. The braiding density of the front-end conical part is greater than that of the rear-end conical part. In order to improve the axial cutting on the thrombus, the front-end conical part is provided with a flat section 12 of 1-2 mm, the flat section 12 is close to the edge of the cylindrical middle part, and the flat section 12 has a better cutting effect on the thrombus than a circular braid wire which is conventionally used. At the same time, the length of the flat section 12 is limited to 1-2 mm, without hindering the overall deformation of the braid wire.

Although the present invention has been described in detail above, the present invention is not limited thereto, and various modifications can be made by those skilled in the art according to the principles of the present invention. Thus, it is intended that the present invention cover the modifications and variations of this invention provided they come within the scope of the appended claims and their equivalents.

1. A safety thrombectomy device, characterized by comprising:
a handle (1);
a hollow outer tube (2);
a hollow inner core tube (3) having a first inner core segment located in a through hole in the outer tube (2), a second inner core segment extending from the front end of the outer tube (2), and a third inner core segment extending from the rear end of the outer tube (2);
a thrombus crusher (5) provided at the front end of the outer tube (2);
a thrombus collector (4) connected to the front end of the second inner core segment of the hollow inner core tube (3) and located in front of the thrombus crusher (5);
a tee joint (6) provided at the front end of the handle (1) and having a free end (10) used for injecting normal saline;
an adjusting knob (7) provided on the handle (1) and capable of moving with respect to the handle (1) to drive the outer tube (2) to move back and forth;
a locking knob (8) provided on the handle (1) and used for locking the adjusting knob (7) after the outer tube (2) moves to a preset position; and
an injector (9) provided on the third inner core segment of the hollow inner core tube (3) provided at the rear end of the handle (1) and used for injecting normal saline.

2. The safety thrombectomy device of claim 1, characterized in that the second inner core segment of the hollow inner core tube (3) extending from the front end of the outer tube (2) passes through the thrombus crusher (5) and the thrombus collector (4) sequentially and is connected to front end of the thrombus collector (4), so that the thrombus crusher (5) and the thrombus collector (4) can be expanded or tightened when the adjusting knob (7) drives the outer tube (2) to move back and forth.

3. The safety thrombectomy device of claim 2, characterized in that the front end of the second inner core segment of the hollow inner core tube (3) is connected to a first hollow metal ring (16) and the front end of the thrombus collector (4) via the first hollow metal ring (16).

4. The safety thrombectomy device of claim 2, characterized in that the outer tube (2) is connected to the tee joint (6) and the adjusting knob (7) via the tee joint (6).

5. The safety thrombectomy device of claim 4, characterized in that the adjusting knob (7) includes a first portion disposed within the handle (1) chute and a second portion extending from the front end of the handle (1) and connected to the tee joint (6).

6. The safety thrombectomy device of claim 2, characterized in that the front end of the thrombus crusher (5) is connected to the rear end of the thrombus collector (4) by a restraining ring (18).

7. The safety thrombectomy device of claim 3, characterized in that the first hollow metal ring (16) is connected to a hollow flexible lead-in head (15) in front of it.

8. The safety thrombectomy device of claim 7, characterized by further comprising a guide wire passing sequentially through the hollow cavity of the hollow inner core tube (3), the hollow cavity of the first hollow metal ring (16), and the hollow cavity of the hollow flexible lead-in head (15).

9. The safety thrombectomy device of claim 1, characterized by the free end (10) of the tee joint (6) is provided with a plugging end.

10. The safety thrombectomy device of claim 1, characterized in that the outer end of the injector (9) is provided with a plugging end.

11. The safety thrombectomy device of claim 10, characterized in that the front end of the thrombus collector (4) is fixedly connected to the front end of the hollow inner core tube (3), the rear end of the thrombus crusher (5) is connected to the front end of the outer tube (2), and the rear end of the thrombus collector (4) is connected to the front end of the thrombus crusher (5) via a restraining ring (18), the thrombus collector (4) and the thrombus crusher (5) are respectively provided with a developing ring (13).

12. The safety thrombectomy device of claim 11, characterized in that the thrombus collector (4) has at least one convex ring (11) at an axially central position of the collecting mesh, the diameter of the convex ring (11) is 0.5-2 mm larger than the diameter of the rest of the collecting mesh.

13. The safety thrombectomy device of claim 12, characterized in that the braiding density of metal wires of the convex rings (11) is greater than the braiding density of the rest of the collecting mesh.

14. The safety thrombectomy device of claim 13, characterized in that the thrombus collector (4) has two convex rings (11) on the collecting mesh.

15. The safety thrombectomy device of claim 14, characterized in that the thrombus crusher (5) has a crushing mesh made of metal braid wires, the structure of the crushing mesh is that the middle is hollow cylindrical part, both ends of the hollow cylindrical part are conical parts, wherein the conical part with higher braiding density has a flat section (12) of 1-2 mm, the flat section (12) is close to the edge of the cylindrical part.
